(19) United States
(12) Patent Application Publication
(10) Pub. No.: US 2010/0125265 A1
(43) Pub. Date: May 20, 2010

(54) Cell Delivery System to Induce Cell Growth and Angiogenesis

(75) Inventors: Noreen Moloney, Moycullen (IE); Angela Duffy, Rahoon (IE)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/274,803

(22) Filed: Nov. 20, 2008

(51) Int. Cl. A61M 25/00

(52) U.S. Cl. 604/522; 604/244

(57) ABSTRACT

Systems and methods that allow the local delivery of stem cells and/or endothelial progenitor cells to a treatment site. More particularly, this disclosure provides cell delivery systems used to induce cell growth and angiogenesis at a treatment site.

### FIELD OF THE INVENTION

The present disclosure relates to systems and methods that allow the local delivery of stem cells and/or endothelial progenitor cells to a treatment site.

### BACKGROUND OF THE INVENTION

Cardiovascular disease or injury can lead to localized tissue damage and can impair blood supply throughout the body. When the blood supply to the heart is compromised, certain cells will respond by generating compounds that induce the growth of new vessels. The process by which these new blood vessels are induced to grow is termed angiogenesis, and the compounds that induce the new growth are referred to as angiogenic factors.

Blood vessels are the means by which oxygen and nutrients are supplied to living tissues and waste products removed from living tissue. Angiogenesis is the process by which new blood vessels are formed, as reviewed, for example, by Folkman and Shing, J. Biol. Chem. 267 (16), 10931-10934 (1992). Thus angiogenesis is a critical process. It is essential in reproduction, development and wound repair. However, inappropriate angiogenesis can have severe consequences. For example, it is only after many solid tumors are vascularized as a result of angiogenesis that the tumors begin to grow rapidly and metastasize. Because angiogenesis is so critical to these functions, it must be carefully regulated in order to maintain health. The angiogenesis process is believed to begin with the degradation of the basement membrane by proteases secreted from endothelial cells (EC) activated by mitogens such as vascular endothelial growth factor (VEGF) and basic fibroblast growth factor (bFGF). The cells migrate and proliferate, leading to the formation of solid endothelial cell sprouts into the stromal space, then, vascular loops are formed and capillary tubes develop with formation of tight junctions and deposition of new basement membrane.

Often, particularly if the tissue damage is too severe, the body's natural angiogenic response is inadequate to deal with the disease or injury at hand. As a result, scientists have begun to look at exogenous angiogenic factors and methods of delivering these factors to the site of disease or injury to supplement the body's response.

Scientists have attempted to increase the physiological stem cell count or to increase the mobilization of stem cells using granulocyte colony stimulation factor (G-CSF). However, these efforts have not been successful and have not resulted in a sufficient accumulation of stem cells at the site of injury to be beneficial. The delivery of an effective amount of bioactive material to a treatment site without producing unwanted systemic side effects has proven to be a significant challenge to the medical and pharmaceutical industries.

Recent research suggests that multiple factors appear to influence long-term cell implant viability, including the site of cell implant, the type of cells used in the implant and the techniques used in the preparation of the cells to be transplanted. Even taking all the above factors into considerations, only about 3-20% of the implanted cells survive for more than seven days. Membrane trauma and other factors related to the delivery device or the method of cell delivery seem to have a significant effect on the attrition rate of transplanted cells. Therefore, improvements to the cellular compositions delivered and to the method of cell delivery could greatly increase the therapeutic efficacy of the treatment.

### SUMMARY OF THE INVENTION

The present disclosure is directed toward systems and methods for the local delivery of stem cells and/or endothelial progenitor cells to a treatment site. Various embodiments of the present disclosure may include a cell delivery system used to induce cell growth and angiogenesis at a treatment site.

In one aspect, the present disclosure provides a cell delivery catheter system to locally induce angiogenesis in the cardiovascular system comprising a hollow shaft with a distal and proximal end, having a tapered nose at the distal end; at least one perfusion hole in the distal region of the hollow shaft, proximal to the tapered nose; a mesh structure inside the lumen of the hollow shaft, proximal to the perfusion hole(s) and perpendicular to the hollow shaft; a sack of liquid proximal to the mesh structure, wherein the liquid comprises a therapeutic cell growth promoting composition; and a plunger proximal to the sack of liquid, wherein the plunger is advanced causing the sack of liquid to burst against the mesh structure and the cell growth promoting composition to filter out of the perfusion hole(s). In another aspect, the hollow shaft comprises a material selected from the group consisting of metal and polymer. In another aspect, the plunger of the catheter system is removable so that the cell growth promoting composition may be inserted into the device.

In different embodiments, the therapeutic cell growth promoting composition may include cells selected from the group consisting of pluripotent stem cells such as embryonic stem and germ cells; multipotent progenitor cells such as hematopoietic stem cells; myoblast; multipotent adult germlne stem cells (maGSCs); totipotent cells, and combinations thereof. In another aspect, the therapeutic cell growth promoting composition may comprise endothelial progenitor cells. In another embodiment, the cell growth promoting compositions may be administered to a point or distal to a point of injury or blockage in the arterial system. In another embodiment, the cell growth promoting compositions may be administered to the pericardial sac.

In another aspect, the present disclosure provides a method for locally inducing angiogenesis comprising the steps of administering a cell delivery catheter system comprising a hollow shaft having a tapered nose at the distal end, at least one perfusion hole present in the distal region of the hollow shaft, proximal to the tapered nose, a mesh structure inside the lumen of the hollow shaft, proximal to the perfusion hole(s) and perpendicular to the hollow shaft, a sack of liquid proximal to the mesh structure, wherein the liquid comprises a therapeutic cell growth promoting composition, and a plunger proximal to the sack of liquid, wherein the plunger is advanced causing the sack of liquid to burst against the mesh structure and the therapeutic cell growth promoting composition to filter out of the perfusion hole(s); delivering a therapeutically effective amount of the therapeutic cell growth promoting composition to a point or distal to a point of injury or blockage in the cardiovascular system; and advancing the plunger and causing the sack of liquid to burst and the therapeutic cell growth promoting composition to filter out of the perfusion hole(s) and into the surrounding cardiovascular tissue.

In one embodiment, the method may include therapeutic cell growth promoting compositions comprising cells selected from the group consisting of multipotent progenitor cells, pluripotent stem cells, and totipotent stem cells. In another embodiment, the therapeutic cell growth promoting compositions may include endothelial progenitor cells. In another embodiment, the cell growth promoting compositions may be delivered to a point or distal to a point of injury or blockage in the arterial system. In another embodiment, the cell growth promoting compositions may be delivered to the pericardial sac.

A typical target site may include the heart or the coronary vasculature of a patient. In one embodiment, the cell delivery catheter system may be used to treat an ischemic or injured heart. In another embodiment, the cell delivery system may be used to treat arterial blockage. In another embodiment, the cell delivery system may be used to treat injuries and cardiovascular diseases such as angioplasty, ischemia, stenosis, cardiomyopathies, coronary artery disease and pericardial diseases.

### BRIEF DESCRIPTION OF THE DRAWINGS

FIG. 1 provides an illustration of one embodiment the disclosed catheter system for inducing angiogenesis.

### DETAILED DESCRIPTION OF THE INVENTION

Presently disclosed are systems and methods for the local delivery of a therapeutic cell growth promoting composition to a treatment site to locally induce angiogenesis, stimulate collateral blood vessel formation, improve function or promote tissue regeneration.

To aid in the understanding of the invention, the following non-limiting definitions apply herein:

The term “effective amount” as applied to cell growth promoting composition means the amount of the compound which is generally sufficient to effect a desired change in the subject. The pharmaceutically effective amount for the purposes disclosed herein depends on the treatment area and the type of cells incorporated in the cell growth promoting composition, and is determined by such considerations as are known in the art.

As used herein, the term “endothelial cell mitogen” means any protein, polypeptide, mutein or portion thereof that is capable of, directly or indirectly, inducing endothelial cell growth and division. Such proteins include, for example, acidic and basic fibroblast growth factors (aFGF and bFGF), vascular endothelial growth factor (VEGF), epidermal growth factor (EGF), transforming growth factor α and β (TGF-α and TFG-β) platelet-derived endothelial growth factor (PD-ECGF), platelet-derived growth factor (PDGF), tumor necrosis factor a (TNF-α), hepatocyte growth factor (HGF), insulin like growth factor (IGF), erythropoietin, colony stimulating factor (CSF), macrophage-CSF (M-CSF), granulocyte/macrophage CSF (GM-CSF) and nitric oxidesynthase (NOS). See, Klagsbrun, et al., Annu. Rev. Physiol., 53:217-239 (1991); Folkman, et al., J. Biol. Chem., 267:10931-10934 (1992) and Symes, et al., Current Opinion in Lipidology, 5:305-312 (1994). Muteins or fragments of a mitogen may be used as long as they induce or promote EC cell growth.

“Local administration” or “local delivery” means administration of a cell growth promoting composition to or to the vicinity of a site intended to receive a beneficial or therapeutic effect of the compositions administered with the systems and methods described herein. Local administration excludes systemic (i.e. blood circulatory system) or oral routes of administration. The vicinity of the site of injury may be the area directly adjacent to the area inured or in close enough proximity to the injured area to enable the therapeutic composition

“Practitioner” means a person who is using the methods and/or kits of the current disclosure on the patient. This term includes, without limitation, doctors, other medical personnel, veterinarians, and scientists.

“Stem cells” as used herein refers to unspecialized cells capable of self-renewal and differentiating into specialized cell types. Stem cells include embryonic stem cells and adult stem cells.

“Therapeutic” or “therapeutically” as used herein refers to

A person skilled in the art will undoubtedly appreciate that at least some part of the prepared suspension will be lost. Accordingly, the term “total volume” refers to the total volume injected to a patient, not a volume of the suspension prepared.

“Treating” or “treatment” means treatment of a disease or injury in a patient by (i) inhibiting the progression of the disease, i.e., arresting its development; or (ii) relieving the disease, i.e., reducing the incidence of symptoms of or causing regression of the disease.

The term “treatment area” “target area” or “treatment site” includes any area that is intended to receive a beneficial or therapeutic effect of the therapeutic composition administered with the systems and methods described herein.

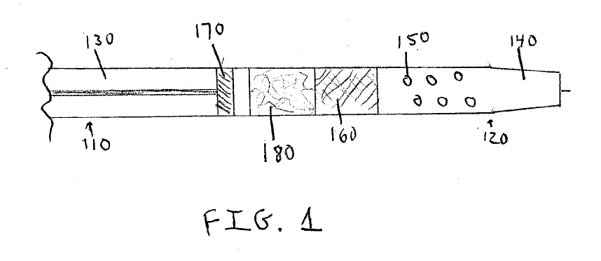

Referring to FIG. 1, one embodiment of the present disclosure comprises a catheter having a proximal 110 and distal end 120, a hollow shaft 130 with a tapered nose 140, at least one perfusion hole 150 proximal to the tapered nose 140, a mesh structure 160 inside the hollow shaft 130 and proximal to the perfusion holes 150, and a plunger 170 proximal to the mesh structure. A sack or pouch filled with liquid comprising a therapeutic cell growth promoting composition (not depicted) may be placed between the mesh structure 160 and the plunger 170, wherein advancing the plunger causes the sack of liquid to burst against the mesh structure and causes the therapeutic cell growth promoting composition to perfuse through the mesh structure and out of the perfusion holes to the target area.

Delivery to the target area can be accomplished using a catheter (e.g. infusion catheter, diagnostic catheter, etc.) In different embodiments, the suspension may be delivered to the targeted area via a catheter and injected to the targeted area through a wall of a blood vessel adjacent to the targeted area. For example, targeting the myocardial tissue, via a TransAccess percutaneous transvenous catheter, cells can be delivered into through the wall of anterior interventricular artery and into the anterior wall of the LV. Other regions such as lateral and posterior myocardium can also be targeted with the mentioned device and use the methods described in this invention. Non-limiting examples of suitable catheters include the infusion catheter, diagnostic catheter and TransAccess catheter delivery system (Medtronic, Inc., Minneapolis, Minn.). In one embodiment, the suitable catheter is Pioneer CX delivery catheter (Medtronic, Inc., Minneapolis, Minn.). In another embodiment, the catheter is a minimally invasive transvenous catheter, such as, for example, TransAccess LT (available from Medtronic, Inc., Minneapolis, Minn.).

The catheter system may comprise a hollow shaft with an inner lumen. The hollow shaft may have a proximal end and a distal end. The distal end may have a tapered nose and at least one perfusion hole proximal to the tapered nose. Preferably, the number of perfusion holes proximal to the tapered nose will be the number necessary to locally deliver an effective amount of the therapeutic composition. Proximal to the perfusion hole(s), the catheter system may comprise a perpendicularly-oriented mesh-like structure inside the lumen of the hollow shaft.

In one embodiment, the hollow shaft may comprise 304V stainless steel. Alternatively, other biocompatible metal materials such as nitinol may be used. In one exemplary embodiment, the present invention may be fabricated from bioabsorbable materials such as poly lactic acid (PLA), polyglycolic acid (PGA), polysebacic acid (PSA), poly(lactic-co-glycolic) acid copolymer (PLGA),poly(lactic-co-sebacic) acid copolymer (PLSA), poly(glycolic-co-sebacic) acid copolymer (PGSA), polyesters, polyorthoesters, polyanhydrides, polyiminocarbonates, inorganic calcium phosphate, aliphatic polycarbonates, polyphosphazenes, collagen based adhesive, fibrin based adhesive, albumin based adhesive, polymers or copolymers of caprolactones, amides, amino acids, acetals, cyanoacrylates, degradable urethanes; or biocompatible but non-bioabsorable materials such as acrylates, ethylene-vinyl acetates, non-degradable urethanes, styrenes, vinyl chlorides, vinyl fluorides, TEFLON® (DuPont, Wilmington, Del.), nylon, HYTREL (DuPont) or PEBAX (Autofina). The above disclosure is not an exhaustive list, but instead represents alternate embodiments illustrated by way of example only. Those of ordinary skill in the art are knowledgeable of and will readily employ the numerous biocompatible, biodegradable and bioerodable materials in the art in order to achieve the spirit of the current invention.

The cells in the cell growth promoting compositions may be selected from the group consisting of pluripotent stem cells such as embryonic stem and germ cells; multipotent progenitor cells such as hematopoietic stem cells and endothelial progenitor cells; mesenchymal stem cells; myoblast; mature myogenic cells (e.g., skeletal myocytes, cardiomyocytes, purkinje cells, fibroblasts); bone marrow stromal cells, multipotent adult germline stem cells (maGSCs); totipotent cells, and combinations thereof.

Sources of cells in the cell growth promoting composition may include embryonic tissue, gonads, epithelial tissue, cardiac tissue, bone marrow, blood, adipose tissue, skeletal tissue, or any other tissue containing stem cells. The cells may be obtained by any suitable method known to a person of ordinary skill in the relevant art.

In one aspect, the therapeutic cell growth promoting composition comprises cells which possess the functions of native cells or cells which can differentiate into suitable types. Such cells may include progenitor myogenic cells (e.g., myoblast), mature myogenic cells (e.g., skeletal myocytes, cardiomyocytes, smooth muscle myocytes, myofibroblast, etc.), mature endothelial cells, bone marrow stromal cells, peripheral blood stem cells, multipotent adult progenitor cells (MAPC), fetal cardiomyocytes, neonatal cardiomyocytes, embryonic stem cells, bone marrow derived angioblasts, CD34+ cells, CD133+ cells, CD117+ cells and combinations thereof.

In one embodiment, to further enhance angiogenesis, an endothelial progenitor cell modified to express an endothelial cell mitogen may be used. In another embodiment, endothelial cell mitogens may be administered to the patient in conjunction with, or subsequent to, the administration of the endothelial progenitor cells. See, Baffour, et al., supra (bFGF); Pu, et al, Circulation, 88:208-215 (1993) (aFGF); Yanagisawa-Miwa, et al., supra (bFGF); Ferrara, et al., Biochem. Biophys. Res. Commun., 161:851-855 (1989) (VEGF); (Takeshita, et al., Circulation, 90:228-234 (1994)).

The therapeutic cell growth promoting composition may also include angiogenesis-related cytokines, such as vascular endothelial growth factor (VEGF) and hepatocyte growth factor (HGF), anti-thrombogenic agents or other agents for suppressing stenosis or late restenosis such as heparin, streptokinase, urokinase, tissue plasminogen activator, anti-thromboxane B agents, anti-B-thromboglobulin, prostoglandin E, aspirin, dipyridimol, anti-thromboxane A2 agents, murine monoclonal antibody 7E3, triazolopyrimidne, ciprostene, hirudin, ticlopidine, nicorandil, and combinations thereof. Anti-platelet derived growth factor may be used as therapeutic agent to suppress subintimal fibromuscular hyperplasia at an arterial stenosis site, or any other inhibitor of cell growth at the stenosis site may be used.

Other therapeutic agents that may be included in the cell growth promoting composition may comprise a vasodilator to counteract vasospasms such as an antispasmodic agent, a vasoactive agent such as calcium antagonists, or alpha and beta andrenergic agonists or antagonists. Additionally, the composition may include a biological adhesive such as medical grade cyanoacrylate adhesive or fibrin glue. Additionally, the composition may include an anti-neoplastic agent and a controlled release carrier. The therapeutic composition may include an antibiotic, which may be applied to an infection localized in the body.

The cells in the therapeutic composition may be delivered with a pharmaceutically acceptable carrier. Examples of pharmaceutically acceptable carriers include but are not limited to McCoy's medium, saline, neutral buffered saline, saline mixed with serum albumin, dextrose, water and combinations thereof. Other acceptable carriers include excipients, lubricants, binders, disintegrants, disintegration inhibitors, absorption promoters, adsorbers, moisturizing agents, solvents, solubilizing agents, suspending agents, isotonic agents, buffers, soothing agents, or a combination thereof.

Acceptable carriers, excipients or stabilizers used in the therapeutic composition may be nontoxic and inert at the dosages and concentrations employed, and may include buffers such as phosphate, citrate, or other organic acids; low molecular weigh peptides; proteins such as serum albumin, recombinant albumin, gelatin, or immunoglobulins; hydrophilic polymers; amino acids; monosaccharides, disaccharides, dextrins, glucose derivatives and other carbohydrates; chelating agents such as EDTA, sugar alcohols such as mannitol or sorbitol; salt-forming counterions such as sodium; nonionic surfactants such as Tween or polyethylene glycol (PEG); or combinations thereof.

Suitable pro-cell survival agents include, without limitation, caspase inhibitors, non-toxic seleno-organic free radical scavengers, estrogen steroid hormones (e.g., 17-[beta]-estradiol, estrone) and structurally related derivative compounds, and any combination thereof.

Suitable non-limiting examples of anti-proliferative agents include enoxaparin, angiopeptin, colchicine, hirudin, paclitaxel, paclitaxel analogues, paclitaxel derivatives, amlodipine, doxazosinand, and any combinations thereof.

A sack or a pouch may be the vehicle that is used to deliver the therapeutic composition to the target or treatment site. The sack or pouch may be manufactured from biocompatible materials that are permeable to the therapeutic agent and composition contained therein. Alternatively, the sack may be manufactured from materials that are impermeable to the therapeutic agent and the composition contained therein.

The sack or pouch can be manufactured from materials such as, but not limited to, polyolefins (polypropylene, polyethylene), polyurethanes, silicones, polyesters (Dacron®) and fluorinated polyolefins (PTFE).

In another embodiment, the sack can be manufactured from materials such as, but are not limited to, synthetic polymers, natural polymers, inorganics or a blend of various compositions, as required. Synthetic polymers include, but are not limited to, Aliphatic polyesters, such as poly(lactic acid), poly(glycolic acid), poly(lactic acid-co-glycolic acid), poly(ε-caprolactone), poly(trimethylene carbonate), polydioxanone and copolymers, poly(hydroxy butyrate) (Biopol®), poly(hydroxy valerate), poly(hydroxy butyrate-co-hydroxy valerate), poly(butylene succinate) (Bionolle®), poly(butylene adipate), polyanhydrides, poly(ortho ester)s, poly(ester amide)s, poly(ester urethane)s, poly(ester anhydride)s, poly(ester carbonate)s, polyphosphazenes, polyarylates, Poly(ether ester)s, polyolefins, polyurethanes, fluorinated polyolefins, chlorinated polyolefins, polyamides, acrylate polymers, such as poly(methyl methacrylate) and copolymers (Eudragit®), Acrylamide polymers, vinyl polymers, polyacetals, polycarbonates, polyethers, aromatic polyesters, poly(ether ether ketone)s, polysulfones, silicone rubbers, thermosets, such as epoxies, and poly(ester imide).

Natural polymers can include, but are not limited to, albumin, collagen, gelatin, hyaluronic acid, starch, alginate, pectin, cellulose and cellulose derivatives (such as methylcellulose, hydroxypropylcellulose, hydroxypropylmethylcellulose, carboxy-methylcellulose, cellulose acetate phthalate, cellulose acetate succinate, hydroxypropylmethylcellulose phthalate), casein, dextran, polysaccharides (such as sucrose acetate isobutyrate), and fibrinogen.

Representative examples of inorganics can include hydroxyapatite, tricalcium phosphate, and silicates, such as Bioglass®, montmorillonite, and mica.

A treatment site or target area is an area which is in need of the treatment provided. For example, a therapeutic cell growth promoting composition is appropriate for areas in need of cellular repopulation. Such need may arise because of multiple reasons, such as, for example, trauma, wounding or infraction. In one embodiment of the present disclosure, the targeted area may be the heart. In another embodiment, the treatment area may be the pericardial sac housing the heart. In another embodiment, the treatment area may be the myocardial tissue. In another embodiment, the treatment area may be at or distal to the point of stenosis in the arterial system.

The amount of the therapeutic composition administered to the subject will depend on the body weight of the subject, the cell growth promoting agent, the overall composition, the volume, the location of administration, the method of delivery and the degree of tissue damage or disease progression. Those skilled in the art can readily determine the appropriate dosage for the selected composition and specific subject.

The methods of introducing the catheter into the blood vessels are known to persons of ordinary skill in the art. In one non-limiting example, the catheter can be introduced into a femoral vein and advanced into the vessel adjacent to the targeted area.

For example, if the vessel adjacent to the targeted area is the anterior interventricular artery, the catheter may be advanced from the femoral vein through the right ventricle to the coronary sinus and then to the great cardiac vein. The catheter then penetrates the great cardiac vein and reaches the anterior interventricular artery. A person of ordinary skill in the art will appreciate that the therapeutic composition may be delivered to the targeted area in more than one injection. The practitioner practicing the current methods would have the ordinary skill of determining the volume to be injected. Another important consideration is the total volume of the suspension which can be injected safely and efficiently. The present invention provides important novel information for both of these considerations.

Alternatively, the cell growth promoting composition may be delivered directly to the myocardium using an infusion catheter, stiletto catheter, balloon catheter, needle, injector, channeling device or other medical device appropriate for delivery into the myocardium. Delivery can be guided by fluoroscopy, echocardiography, MRI, or electromecahnical mapping. Alternatively, transendocardial or transepicardial approaches may be necessary, as determined by the practitioner.

It is recognized that the length and internal diameter of the catheter may affect the fluid dynamics and delivery of the therapeutic composition. Various catheter lengths, internal diameters and shear rates can be used with selected cell density solutions and may depend on a particular measure of effective delivery.

Particular methods of the present invention may be used to treat blood vessel injuries that result in denuding of the endothelial lining of the vessel wall. For example, primary angioplasty is becoming widely used for the treatment of acute myocardial infarction. In addition, endovascular stents are becoming widely used as an adjunct to balloon angioplasty. However, the liability of the endovascular prosthesis has been its susceptibility to thrombotic occlusion in approximately 3% of patients with arteries 3.3 mm or larger. If patients undergo stent deployment in arteries smaller than this the incidence of sub-acute thrombosis is even higher. Sub-acute thrombosis is currently prevented only by the aggressive use of anticoagulation. The combination of vascular intervention and intense anticoagulation creates significant risks with regard to peripheral vascular trauma at the time of the stent/angioplasty procedure and therefore could create a condition wherein induced angiogenesis would be beneficiary.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

1: A catheter system for delivering cells to locally induce angiogenesis in the heart or arterial system comprising:
(a) a catheter having a proximal end, a distal end, a hollow shaft and a tapered nose at said distal end;
(b) at least one perfusion hole present proximal to said tapered nose;
(c) a mesh structure inside said hollow shaft, proximal to said perfusion hole and perpendicularly-oriented to said hollow shaft;
(d) a sack of liquid proximal to said mesh structure, wherein said liquid comprises a therapeutic cell growth promoting composition; and
(e) a plunger proximal to said sack of liquid, wherein said plunger is advanced causing said sack of liquid to burst against said mesh structure and to filter out of said mesh structure and perfuse out of said hollow shaft through said perfusion hole to target area.

2: The catheter system of claim 1, wherein said hollow shaft comprises a material selected from a metal, a polymer or a combination thereof.

3: The catheter system of claim 1, wherein said plunger can be removed to allow said cell sack of liquid to be inserted into said hollow shaft.

4: The catheter system of claim 1, wherein said therapeutic cell growth promoting composition comprises cells selected from multipotent progenitor cells, pluripotent stem cells, totipotent stem cells, or a combination thereof.

5: The catheter system of claim 1, wherein said therapeutic cell growth promoting composition comprises endothelial progenitor cells.

6: The catheter system of claim 1, wherein said therapeutic cell growth promoting composition is administered near or to a point of injury or blockage in the arterial system.

7: The delivery system of claim 1, wherein said cell growth promoting composition is administered to the pericardial sac.

8: A method for locally inducing angiogenesis comprising the steps of:
(a) providing a cell delivery catheter system comprising:
(1) a catheter having a proximal end, a distal end, a hollow shaft and a tapered nose at said distal end;
(2) at least one perfusion hole present proximal to said tapered nose;
(3) a mesh structure inside said hollow shaft, proximal to said perfusion hole and perpendicularly-oriented to said hollow shaft;
(4) a sack of liquid proximal to said mesh structure, wherein said liquid comprises a therapeutic cell growth promoting composition; and
(5) a removable plunger proximal to said sack of liquid;
(b) advancing said plunger and causing said sack of liquid to burst against said mesh structure and said therapeutic cell growth promoting composition to filter out of said mesh structure and perfuse out said hollow shaft through said perfusion hole to target area; and
(c) delivering a therapeutically effective amount of said therapeutic cell growth promoting composition near or to a point of injury or blockage in the cardiovascular system, thereby locally inducing angiogenesis.

9: The method of claim 8, wherein said sack of liquid can be loaded into said catheter at any point before or during treatment.

10: The method of claim 8, wherein said cell growth promoting composition comprises cells selected from the group consisting of multipotent progenitor cells, pluripotent stem cells, and totipotent stem cells.

11: The method of claim 8, wherein said cell growth promoting composition comprises endothelial progenitor cells.

12: The method of claims 8, wherein said cell growth promoting composition is delivered to a point or distal to a point of injury or blockage in the arterial system.

13: The method of claims 8, wherein said cell growth promoting composition is delivered to the pericardial sac.
